(19) United States
(12) Patent Application Publication
(10) Pub. No.: US 2012/0171156 A1
(43) Pub. Date: Jul. 5, 2012

(54) METHODS AND COMPOUNDS FOR REDUCING ALLERGIC REACTIONS TO HAIR DYES

(76) Inventors: David Basketter, Sharnbrook (GB); John McFadden, London (GB); Ian White, Gerrards Cross (GB)

(21) Appl. No.: 13/379,275

(22) PCT Filed: Jun. 17, 2010
(86) PCT No.: PCT/EP2010/058593

§ 371 Date: Mar. 20, 2012

Related U.S. Application Data
(60) Provisional application No. 61/223,241, filed on Jul. 6, 2009.

(30) Foreign Application Priority Data
Jun. 19, 2009 (GB) 0910629.5

(51) Int. Cl. A61K 31/355; A61K 36/9062; A61K 35/74; A61K 36/82; A61P 17/00; A61K 33/04; A61K 33/24; A61K 38/44; A61P 37/08; A61K 36/48; A61K 31/765

(52) U.S. Cl. 424/78.37; 514/458; 424/757; 424/756; 424/115; 424/729; 424/711; 424/710; 424/722; 424/650; 424/94.4

(57) ABSTRACT

Conventional commercial hair dyes form potent allergens and may also have irritant properties. If they were not already well established in the market and the side effects tolerated by consumers they would likely be deemed unsafe for use. The invention relates to compounds, compositions, formulations and methods for reducing, avoiding or eliminating allergic contact dermatitis reactions to conventional commercial hair dyes.

The invention relates to compounds, compositions, formulations and methods for: avoiding; eliminating; or reducing ie inhibiting the induction and/or decreasing the severity and/or frequency of elicitation of allergic reactions to generic hair dyes.

Hair dyeing products include all compounds, compositions formulations and methods for colouring, tinting or highlighting the hair.

Existing hair dye products often contain potent allergens and may also have irritant properties. If these hair dye products were not already well established in the market they may well be deemed too harmful for use by hairdressers or consumers.

Normally an individual only becomes sensitised after one, or more usually, after several exposures to the hair dye product. Hence, hair dye products which are initially well tolerated may, after further use, cause an allergic reaction.

The frequency of allergic reactions to hair dye products has not been measured with great precision in the general population, but it is common (McFadden J P. (2008). Allergy and consumer products; what constitutes an epidemic? Contact Dermatitis 59:325-326; and Thyssen J P and White J M L. (2008) Epidemiological data on consumer allergy to p-phenylenediamine. Contact Dermatitis 59,327-343).

For example, in Thailand, measurement of paraphenylenediamine (PPD) skin contact allergy, a key indicator for hair dye allergy, in their general population would suggest that more than a million adults have this problem (Basketter D A et al, (2006) The impact of exposure variables on the induction of skin sensitization. Contact Dermatitis, 55, 178-185). Estimates from Europe indicate a similar prevelence.

A classical description of hair dye contact allergy was provided by Cronin E. (1980) Contact Dermatitis Churchill Livingstone, Edinburgh, pp 120-122 “dermatitis may begin within a few hours of the dye being used or may appear the following day. The severity of the eczema varies considerably. There may be intense oedema of the face, particularly of the eyes, with exudation of the scalp; erythema and swelling may extend down the neck onto the upper chest and upper arms, and can even become generalised.”

Less frequently, hair dye allergy may cause swelling of the eyes as the main symptom, and the individual may fail to connect their symptoms with hair dyeing. Occasionally there is nothing more than a mild allergic reaction around the scalp margin, but further exposure to the hair dye may cause such mild symptoms to become more severe.

“The severity of PPD reactions ranges from mild erythema at the hairline or ears to swelling of the eyelids and face, accompanied by an acute vesicular eruption in the scalp that requires prompt medical attention.” (Rietschel R L and Fowler J F. (2008) Hair coloring preparations. Fisher's Contact Dermatitis, B C Decker, Hamilton, 6^{th} edition, pp 303-304).

It has also been observed that reactions in skin sites adjacent to the scalp, such as facial swelling and inflammation, are commoner than scalp related symptoms. As a result cases can be mistaken for angioedema (facial swelling from for example an ingested allergen, such as peanut) (Søsted H, Agner T Andersen K E and Menné T. (2002) 55 cases of allergic reactions to hair dye: a descriptive, consumer complaint-based study. Contact Dermatitis, 47: 299-303).

A unifying feature of allergic reactions to hair dyes is that the reactions tend to be more prominent on the skin around the scalp (neck, face, ears) than on the scalp itself, this only tending to be involved in the more severe cases. This may be readily explained as any chemical applied to hair-bearing skin will largely be absorbed onto the large surface area provided by the hair, thus reducing the dose available for exposure on the skin surface.

Hairdressers are at particular risk of developing allergic reactions to hair dyes on their hands as a result of repeated exposure to hair dyeing products in the course of their work.

The present invention aims to reduce, avoid or eliminate both the induction (sensitisation) of contact allergy and/or the elicitation of allergic contact dermatitis in individuals exposed to hair dye products.

In a first aspect the invention relates to a method of reducing induction of contact allergy or reducing elicitation of allergic contact dermatitis of, in or on the skin in response to hair dye comprising inhibiting oxidation of the hair dye.

In a second aspect the invention relates to a method wherein oxidation is inhibited or reversed using one or more of: antioxidant, reducing agent, and a neutralising system. In another aspect the invention relates to a method wherein the antioxidant is selected from vitamin E and vitamin E derivatives.

In another aspect the invention relates to a method wherein the antioxidant is selected from organic esters, fatty acids, vitamin E acetate and vitamin E linoleate.
In another aspect the invention relates to a method wherein the antioxidant is selected from 2-acetylhydroquinone, 4-butylresorcinol, acacia victoriae fruit extract, acer palmatum leaf extract, acetamidocaproic acid, acetyl benzoyloxy prasterone, acetyl cysteine, acetyl trihexyl citrate, adamantanyl hydroxylterephthalamide, adamantanylcarboxamido hydroxylbenzamide, adamantly methylhydroxylterephthalamide, adamantylcarboxamido methylhydroxylbenzamide, agrimonia eupatoria root extract, alchemilla vulgaris leaf extract, allantoin ascorbate, alpha-arbutin, alpinia uraiensis stalk/leaf water, aminoethanesulfinic acid, aminopropyl ascorbyl phosphate, aminopropyl tocopheryl phosphate, anserine, arabidopsis thaliana extract, arbutin, arctium lappa fruit extract, argon, asarum heterotropoides rhizome extract, ascorbic acid, ascorbic acid polypeptide, ascorbyl dipalmitate, ascorbyl glucoside, ascorbyl linoleate, ascorbyl methylsilanol pectinate, ascorbyl palmitate, ascorbyl stearate, ascorbyl tetraisopalmitate, ascorbyl tocopheryl maleate, asiaticoside, avena sativa kernel extract, bacillus/rice bran extract/soybean extract ferment filtrate, benzoguanamine, beta vulgaris root powder, bha, bht, bis-demethoxycurcumin, bis-hydroxyethyl tocopherylsuccinoylamido hydroxypropane, bromphenol blue, buddleja axillaris leaf extract, butylated xylenol, caffeic acid, calcium ascorbate, calophyllum inophyllum seed oil, camellia sinensis catechins, camellia sinensis leaf extract camellia sinensis leaf oil, capparis moonii fruit extract, capsicum annuum fruit extract, carnosic acid, carotenoids, cayaponia tayuya root extract, cercis chinensis flower/leaf/stem extract, chaenomeles sinensis fruit extract, chitosan ascorbate, chitosan glycolate, chitosan salicylate, chlorogenic acids, chrysanthemum boreale flower extract, cimicifuga dahurica root extract, cimicifuga racemosa root extract, cinnamomum zeylanicum bark extract, citrus junos seed extract, citrus medica vulgaris fruit extract, cobalt dna, copper adenosine triphosphate, coptis chinensis root extract, cordyceps sinensis extract, curcumin, cyamopsis tetragonoloba symbiosome extract, cyclopia genistoides leaf extract, cysteamine HCl, cysteine, cysteine HCl, davidsonia pruriens fruit extract, decyl mercaptomethylimidazole, demethoxycurcumin, di-t-butylhydroquinone, diacetylcurcumin, diammonium edta, diamylhydroquinone, dibenzothiophene, dibutylene tetrafurfural, dicetyl thiodipropionate, dicyclopentadiene/t-butylcresol copolymer, digalloyl trioleate, dilauryl thiodipropionate, dimethoxy di-p-cresol, dimethylmethoxy chromanol, dimyristyl thiodipropionate, dioleyl tocopheryl methylsilanol, diosmine, diospyros kaki calyx extract, diretinyl ether, disodium ascorbyl sulfate, disodium rutinyl disulfate, disodium salicylphosphate, disodium ubiquinone, distearyl thiodipropionate, ditridecyl thiodipropionate, dodecyl gallate, dunaliella bardawil powder, epigallocatechin gallate, epimedium sagittatum extract, ergothioneine, eriobotrya japonica leaf protoplast extract, eriocaulon buergarianum extract, erythorbic acid, ethyl ferulate, ethylbisiminomethylguaiacol manganese chloride, ethylhexyl ferulate, ethylhexyl gallate, eucommia ulmoides bark extract, forsythia suspensa fruit extract, fragilaria pinnata extract, furfuryl palmitate, gallic acid, ginkgo biflavones, ginkgo leaf terpenoids, glucosylrutin, glyceryl chromonyl ether, glycyrrhiza glabra root water, haematococcus pluvialis extract, haematococcus pluvialis oil, haematococcus pluvialis powder, hesperetin, hesperetin laurate, hesperidin methyl chalcone, histidine hydrochloride, honokiol, hydrolyzed aspergillus/ginseng extract ferment, hydrolyzed gardenia florida extract, hydrolyzed proanthocyanidin, hydrolyzed soy extract, hydroxy benzoxathiolone, hydroxydecyl ubiquinone, hydroxylamine HCl, hydroxylamine sulfate, inositol hexaniacinate hexaascorbate, isoquercitrin, kaempferol, kojic acid, kojyl glucoside, kojyl methylenedioxycinnamate, larrea divaricata extract, lens culinaris symbiosome extract, ligusticum striatum root extract, linseed oil ascorbate esters, lotus japonicus symbiosome extract, lycium chinense fruit extract, lycopene, lysine hydrochloride, madecassoside, magnesium ascorbate, magnesium ascorbate/pca, magnesium ascorbyl phosphate, magnolol, malachite extract, malpighia punicifolia fruit extract, manganese adenosine triphosphate, manganese dioxide, matteuccia struthiopteris extract, melaleuca alternifolia leaf oil, melatonin, melia azadirachta conditioned media/culture, methoxy PEG-7 acorbic acid, methoxytrimethylphenyl dihydroxyphenyl propanol, methyl di-t-butyl hydroxyhydrocinnamate, methylene di-t-butylcresol, methylsilanol ascorbate, murraya exotica leaf extract, niacinamide hydroxybenzoate, niacinamide thioctate, nordihydroguaiaretic acid, o-tolyl biguanide, octadecyl di-t-butyl-4-hydroxyhydrocinnamate, octanicotinoyl epigallocatechin gallate, olea europaea bud extract, olea europaea fruit unsaponifiables, oxycoccus palustris seed oil, p-hydroxyanisole, paeonol, palmitoyl grapevine shoot extract, PEG/PPG-100/70 tocopheryl ether, PEG/PPG-2/5 tocopheryl ether, PEG/PPG-30/10 tocopheryl ether, PEG/PPG-5/10 tocopheryl ether, PEG/PPG-5/20 tocopheryl ether, PEG/PPG-5/30 tocopheryl ether, PEG/PPG-50/20 tocopheryl ether, PEG/PPG-70/30 tocopheryl ether, pentaerythrityl tetra-di-t-butyl hydroxyhydrocinnamate, perilla ocymoides seed extract, perillyl alcohol, phenylthioglycolic acid, pikea robusta extract, pinus densiflora extract, pinus pinaster bark/bud extract, pinus radiata bark extract, piper nigrum seed, piper umbellatum root extract, pisum sativum symbiosome extract, platycodon grandiflorum root extract, pogostemon cablin leaf/stem extract, polygonum cuspidatum root extract, porphyridium polysaccharide, potassium ascorbyl tocopheryl phosphate, PPG-10 tocophereth-30, PPG-2 tocophereth-5, PPG-20 tocophereth-50, PPG-30 tocophereth-70, PPG-5 tocophereth-2, PPG-5 tocopheryl ether, PPG-70 tocophereth-100, propyl gallate, prunus cerasus extract, prunus cerasus fruit extract, prunus cerasus leaf extract, prunus salicina fruit extract, prunus spinosa wood extract, pyridoxine hydroxybenzoate, pyridoxine hydroxycitrate, pyridoxine salicylate, pyridoxine serinate, quercetin, quercetin caprylate, resacetophenone, resveratrol, retynil formyl aspartamate, rhodochrosite extract, rosa davurica bud extract, rosmarinic acid, rosmarinus officinalis flower extract, rubus arcticus fruit extract, rubus chamaemorus fruit extract, rutin, salnacedin, salvia officinalis extract, sambucus nigra bud extract, santalum acuminatum fruit extract, sargassum pallidum extract, sarothamnus scoparius extract, sedum rosea root extract, sideritis syriaca extract, smithsonite extract, sodium ascorbate, sodium ascorbyl phosphate, sodium ascorbyl/cholesteryl phosphate, sodium bisulfite, sodium erythorbate, sodium metabisulfite, sodium phosphono-pyridoxylidenerhodanine, sodium thioglycolate, sodium tocopheryl phosphate, sodium zinc histidine dithiooctanamide, sorbityl furfural, sponge extract, stearyl gallate, succinoyl ascorbate pentapeptide-6, superoxide dismutase, syzygium leuhmanii fruit extract, t-butylbenzamido hydroxylbenzamide, t-butylbenzamido hydroxylphenylacetamide, t-butylbenzamido methylhydroxylbenzamide, tbhq, telmesteine, terminalia ferdinandiana fruit extract, tetrabutyl ethylidenebisphenol, tetrabutyl phenyl hydroxybenzoate, tetrahexyldecyl ascorbate, tetrahydrobisdemethoxydiferuloylmethane, tetrahydrocurcumin diacetate, tetrahydrodemethoxydiferuloylmethane, tetrahydrodiferuloylmethane, tetramethylchromanol glucosides, thioctic acid, thiodiglycol, thiodiglycolamide, thiodiglycolic acid, thioglycolic acid, thiolactic acid, thiomorpholinone, thiosalicylic acid, thiotaurine, thioxanthine, thymol trimethoxycinnamate, tococysteamide, tocophereth-10, tocophereth-12, tocophereth-18, tocophereth-5, tocophereth-50, tocopherol, tocophersolan, tocopheryl acetate, tocopheryl dimethylglycinate HCl, tocopheryl linoleate, tocopheryl linoleate/oleate, tocopheryl nicotinate, tocopheryl retinoate, tocopheryl succinate, tocoquinone, totarol, tremella fuciformis sporocarp extract, triethyl citrate, trifolium pratense leaf extract, trifolium pratense symbiosome extract, trifolium repens extract, trigonella foenum symbiosome extract, tripropylene glycol, tris(nonylphenyl)phosphite, trisodium ascorbyl isopalmitate phosphate, trisodium ascorbyl palmitate phosphate, trisodium fructose diphosphate, trisodium resveratrol triphosphate, tyrosyl histidine HCl, ubiquinol, ubiquinone, vaccinium myrtillus bud extract, vaccinium myrtillus stem extract, vaccinium vitis-idaea fruit extract, vacciniumvitis-idaea leaf protoplasts, vaccinium vitis-idaea seed oil, vicia sativa symbiosome extract, vitis vinifera fruit powder, vitis vinifera juice, vitis vinifera juice extract, vitis vinifera seed extract, vitis vinifera skin extract, vitis vinifera skin powder, xylyl dibutylbenzofuranone, zinc adenosine triphosphate, zinc ascorbate, zinc dibutyldithiocarbamate and zinc thiosalicylate.

In another aspect the invention relates to a method wherein the reducing agent is selected from aminoethanesulfinic acid, ammonium bisulfite, ammonium sulfate, ammonium sulfite, ammonium thiocyanate, ammonium thioglycolate, ammonium thiolactate, calcium thioglycolate, cysteamine HCl, cysteine cysteine HCl, dihydroxyacetone, dithiodiglycolic acid, dithiothreitol, ethanolamine dithiodiglycolate, ethanolamine thioglycolate, ethyl thioglycolate, glutathione, glyceryl thiopropionate, histidine hydrochloride, lysine hydrochloride, magnesium thioglycolate, mercaptopropionic acid, ninhydrin, p-hydroxyanisole, potassium metabisulfite, potassium sulfite, potassium, tetrathionate, sodium bisulfite, sodium hydrosulfite, sodium hydroxymethane sulfonate, sodium metabisulfite, sodium thioglycolate, sodium thiosulfate, sodium tocopheryl phosphate, stannous chloride, strontium thioglycolate, superoxide dismutase, thiodiglycolic acid, thioglycerin, thioglycolic acid, thiolactic acid, thiosalicylic acid and zinc formaldehyde sulfoxylate.

In another aspect the invention relates to a method wherein the skin is pre-treated to inhibit oxidation prior to the application of the hair dye.

In another aspect the invention relates to a method wherein the pre-treatment is confined to non-scalp skin.

In another aspect the invention relates to a method wherein the skin is post-treated to inhibit or reverse oxidation after the application of the hair dye.

In another aspect the invention relates to a method wherein the post-treatment is confined to non-scalp skin.

In another aspect the invention relates to a composition for reducing induction of contact allergy or reducing elicitation of allergic contact dermatitis of skin to hair dye comprising one or more of: antioxidants, reducing agents, and a neutralising system.

In another aspect the invention relates to a composition wherein the antioxidant is selected from vitamin E, vitamin E derivatives, organic esters, fatty acids, vitamin E acetate, vitamin E linoleate, 2-acetylhydroquinone, 4-butylresorcinol, acacia victoriae fruit extract, acer palmatum leaf extract, acetamidocaproic acid, acetyl benzoyloxy prasterone, acetyl cysteine, acetyl trihexyl citrate, adamantanyl hydroxylterephthalamide, adamantanylcarboxamido hydroxylbenzamide, adamantly methylhydroxylterephthalamide, adamantylcarboxamido methylhydroxylbenzamide, agrimonia eupatoria root extract, alchemilla vulgaris leaf extract, allantoin ascorbate, alpha-arbutin, alpinia uraiensis stalk/leaf water, aminoethanesulfinic acid, aminopropyl ascorbyl phosphate, aminopropyl tocopheryl phosphate, anserine, arabidopsis thaliana extract, arbutin, arctium lappa fruit extract, argon, asarum heterotropoides rhizome extract, ascorbic acid, ascorbic acid polypeptide, ascorbyl dipalmitate, ascorbyl glucoside, ascorbyl linoleate, ascorbyl methylsilanol pectinate, ascorbyl palmitate, ascorbyl stearate, ascorbyl tetraisopalmitate, ascorbyl tocopheryl maleate, asiaticoside, avena sativa kernel extract, bacillus/rice bran extract/soybean extract ferment filtrate, benzoguanamine, beta vulgaris root powder, bha, bht, bis-demethoxycurcumin, bis-hydroxyethyl tocopherylsuccinoylamido hydroxypropane, bromphenol blue, buddleja axillaris leaf extract, butylated xylenol, caffeic acid, calcium ascorbate, calophyllum inophyllum seed oil, camellia sinensis catechins, camellia sinensis leaf extract camellia sinensis leaf oil, capparis moonii fruit extract, capsicum annuum fruit extract, carnosic acid, carotenoids, cayaponia tayuya root extract, cercis chinensis flower/leaf/stem extract, chaenomeles sinensis fruit extract, chitosan ascorbate, chitosan glycolate, chitosan salicylate, chlorogenic acids, chrysanthemum boreale flower extract, cimicifuga dahurica root extract, cimicifuga racemosa root extract, cinnamomum zeylanicum bark extract, citrus junos seed extract, citrus medica vulgaris fruit extract, cobalt dna, copper adenosine triphosphate, coptis chinensis root extract, cordyceps sinensis extract, curcumin, cyamopsis tetragonoloba symbiosome extract, cyclopia genistoides leaf extract, cysteamine HCl, cysteine, cysteine HCl, davidsonia pruriens fruit extract, decyl mercaptomethylimidazole, demethoxycurcumin, di-t-butylhydroquinone, diacetylcurcumin, diammonium edta, diamylhydroquinone, dibenzothiophene, dibutylene tetrafurfural, dicetyl thiodipropionate, dicyclopentadiene/t-butylcresol copolymer, digalloyl trioleate, dilauryl thiodipropionate, dimethoxy di-p-cresol, dimethylmethoxy chromanol, dimyristyl thiodipropionate, dioleyl tocopheryl methylsilanol, diosmine, diospyros kaki calyx extract, diretinyl ether, disodium ascorbyl sulfate, disodium rutinyl disulfate, disodium salicylphosphate, disodium ubiquinone, distearyl thiodipropionate, ditridecyl thiodipropionate, dodecyl gallate, dunaliella bardawil powder, epigallocatechin gallate, epimedium sagittatum extract, ergothioneine, eriobotrya japonica leaf protoplast extract, eriocaulon buergarianum extract, erythorbic acid, ethyl ferulate, ethylbisiminomethylguaiacol manganese chloride, ethylhexyl ferulate, ethylhexyl gallate, eucommia ulmoides bark extract, forsythia suspensa fruit extract, fragilaria pinnata extract, furfuryl palmitate, gallic acid, ginkgo biflavones, ginkgo leaf terpenoids, glucosylrutin, glyceryl chromonyl ether, glycyrrhiza glabra root water, haematococcus pluvialis extract, haematococcus pluvialis oil, haematococcus pluvialis powder, hesperetin, hesperetin laurate, hesperidin methyl chalcone, histidine hydrochloride, honokiol, hydrolyzed aspergillus/ginseng extract ferment, hydrolyzed gardenia florida extract, hydrolyzed proanthocyanidin, hydrolyzed soy extract, hydroxy benzoxathiolone, hydroxydecyl ubiquinone, hydroxylamine HCl, hydroxylamine sulfate, inositol hexaniacinate hexaascorbate, isoquercitrin, kaempferol, kojic acid, kojyl glucoside, kojyl methylenedioxycinnamate, larrea divaricata extract, lens culinaris symbiosome extract, ligusticum striatum root extract, linseed oil ascorbate esters, lotus japonicus symbiosome extract, lycium chinense fruit extract, lycopene, lysine hydrochloride, madecassoside, magnesium ascorbate, magnesium ascorbate/pca, magnesium ascorbyl phosphate, magnolol, malachite extract, malpighia punicifolia fruit extract, manganese adenosine triphosphate, manganese dioxide, matteuccia struthiopteris extract, melaleuca alternifolia leaf oil, melatonin, melia azadirachta conditioned media/culture, methoxy PEG-7 acorbic acid, methoxytrimethylphenyl dihydroxyphenyl propanol, methyl di-t-butyl hydroxyhydrocinnamate, methylene di-t-butylcresol, methylsilanol ascorbate, murraya exotica leaf extract, niacinamide hydroxybenzoate, niacinamide thioctate, nordihydroguaiaretic acid, o-tolyl biguanide, octadecyl di-t-butyl-4-hydroxyhydrocinnamate, octanicotinoyl epigallocatechin gallate, olea europaea bud extract, olea europaea fruit unsaponifiables, oxycoccus palustris seed oil, p-hydroxyanisole, paeonol, palmitoyl grapevine shoot extract, PEG/PPG-100/70 tocopheryl ether, PEG/PPG-2/5 tocopheryl ether, PEG/PPG-30/10 tocopheryl ether, PEG/PPG-5/10 tocopheryl ether, PEG/PPG-5/20 tocopheryl ether, PEG/PPG-5/30 tocopheryl ether, PEG/PPG-50/20 tocopheryl ether, PEG/PPG-70/30 tocopheryl ether, pentaerythrityl tetra-di-t-butyl hydroxyhydrocinnamate, perilla ocymoides seed extract, perillyl alcohol, phenylthioglycolic acid, pikea robusta extract, pinus densiflora extract, pinus pinaster bark/bud extract, pinus radiata bark extract, piper nigrum seed, piper umbellatum root extract, pisum sativum symbiosome extract, platycodon grandiflorum root extract, pogostemon cablin leaf/stem extract, polygonum cuspidatum root extract, porphyridium polysaccharide, potassium ascorbyl tocopheryl phosphate, PPG-10 tocophereth-30, PPG-2 tocophereth-5, PPG-20 tocophereth-50, PPG-30 tocophereth-70, PPG-5 tocophereth-2, PPG-5 tocopheryl ether, PPG-70 tocophereth-100, propyl gallate, prunus cerasus extract, prunus cerasus fruit extract, prunus cerasus leaf extract, prunus salicina fruit extract, prunus spinosa wood extract, pyridoxine hydroxybenzoate, pyridoxine hydroxycitrate, pyridoxine salicylate, pyridoxine serinate, quercetin, quercetin caprylate, resacetophenone, resveratrol, retynil formyl aspartamate, rhodochrosite extract, rosa davurica bud extract, rosmarinic acid, rosmarinus officinalis flower extract, rubus arcticus fruit extract, rubus chamaemorus fruit extract, rutin, salnacedin, salvia officinalis extract, sambucus nigra bud extract, santalum acuminatum fruit extract, sargassum pallidum extract, sarothamnus scoparius extract, sedum rosea root extract, sideritis syriaca extract, smithsonite extract, sodium ascorbate, sodium ascorbyl phosphate, sodium ascorbyl/cholesteryl phosphate, sodium bisulfite, sodium erythorbate, sodium metabisulfite, sodium phosphono-pyridoxylidenerhodanine, sodium thioglycolate, sodium tocopheryl phosphate, sodium zinc histidine dithiooctanamide, sorbityl furfural, sponge extract, stearyl gallate, succinoyl ascorbate pentapeptide-6, superoxide dismutase, syzygium leuhmanii fruit extract, t-butylbenzamido hydroxylbenzamide, t-butylbenzamido hydroxylphenylacetamide, t-butylbenzamido methylhydroxylbenzamide, tbhq, telmesteine, terminalia ferdinandiana fruit extract, tetrabutyl ethylidenebisphenol, tetrabutyl phenyl hydroxybenzoate, tetrahexyldecyl ascorbate, tetrahydrobisdemethoxydiferuloylmethane, tetrahydrocurcumin diacetate, tetrahydrodemethoxydiferuloylmethane, tetrahydrodiferuloylmethane, tetramethylchromanol glucosides, thioctic acid, thiodiglycol, thiodiglycolamide, thiodiglycolic acid, thioglycolic acid, thiolactic acid, thiomorpholinone, thiosalicylic acid, thiotaurine, thioxanthine, thymol trimethoxycinnamate, tococysteamide, tocophereth-10, tocophereth-12, tocophereth-18, tocophereth-5, tocophereth-50, tocopherol, tocophersolan, tocopheryl acetate, tocopheryl dimethylglycinate HCl, tocopheryl linoleate, tocopheryl linoleate/oleate, tocopheryl nicotinate, tocopheryl retinoate, tocopheryl succinate, tocoquinone, totarol, tremella fuciformis sporocarp extract, triethyl citrate, trifolium pratense leaf extract, trifolium pratense symbiosome extract, trifolium repens extract, trigonella foenum symbiosome extract, tripropylene glycol, tris(nonylphenyl)phosphite, trisodium ascorbyl isopalmitate phosphate, trisodium ascorbyl palmitate phosphate, trisodium fructose diphosphate, trisodium resveratrol triphosphate, tyrosyl histidine HCl, ubiquinol, ubiquinone, vaccinium myrtillus bud extract, vaccinium myrtillus stem extract, vaccinium vitis-idaea fruit extract, vacciniumvitis-idaea leaf protoplasts, vaccinium vitis-idaea seed oil, vicia sativa symbiosome extract, vitis vinifera fruit powder, vitis vinifera juice, vitis vinifera juice extract, vitis vinifera seed extract, vitis vinifera skin extract, vitis vinifera skin powder, xylyl dibutylbenzofuranone, zinc adenosine triphosphate, zinc ascorbate, zinc dibutyldithiocarbamate and zinc thiosalicylate.

In another aspect the invention relates to a composition wherein the reducing agent is selected from aminoethanesulfinic acid, ammonium bisulfite, ammonium sulfate, ammonium sulfite, ammonium thiocyanate, ammonium thioglycolate, ammonium thiolactate, calcium thioglycolate, cysteamine HCl, cysteine cysteine HCl, dihydroxyacetone, dithiodiglycolic acid, dithiothreitol, ethanolamine dithiodiglycolate, ethanolamine thioglycolate, ethyl thioglycolate, glutathione, glyceryl thiopropionate, histidine hydrochloride, lysine hydrochloride, magnesium thioglycolate, mercaptopropionic acid, ninhydrin, p-hydroxyanisole, potassium metabisulfite, potassium sulfite, potassium, tetrathionate, sodium bisulfite, sodium hydrosulfite, sodium hydroxymethane sulfonate, sodium metabisulfite, sodium thioglycolate, sodium thiosulfate, sodium tocopheryl phosphate, stannous chloride, strontium thioglycolate, superoxide dismutase, thiodiglycolic acid, thioglycerin, thioglycolic acid, thiolactic acid, thiosalicylic acid and zinc formaldehyde sulfoxylate.

### DETAILED DESCRIPTION

Commercial hair dyes for application to/by consumers contain a mixture of organic chemicals which, when exposed to an oxidation system, undergo a variety of coupling reactions to form the coloured chemicals required to generate the wide range of shades desired by consumers.

An undesirable consequence of the generation of a number of chemically reactive intermediates is that these can, during the formation of the coloured chemicals, combine with skin proteins, rendering them “foreign” and thus activating the immune system into the production of an allergic reaction ie a cell mediated, Type IV hypersensitivity.

Allergic contact dermatitis arises during the elicitation phase (clinical reaction in someone already sensitised or made allergic).

An active sensitisation phase (induction) is required before an allergic response can be elicited. Typically an individual only becomes sensitised to an allergenic hair dye following repeated exposures. The number of exposures required for sensitisation to occur is highly variable, the frequency of hair dyeing may mean a period of many months or years precedes the acquisition of sensitisation. Many individuals will never become sensitised.

The precise chemical nature of the reactive intermediates produced by oxidation and which of these causes allergy is the subject of continuing study. However, it is clear that primary agents are oxidation products of aromatic amines such as PPD.

PPD is a well recognized and common cause of allergic contact dermatitis. It is the primary agent used for the diagnosis of hair dye allergy. Here it is used as an indicative example of oxidative hair dye allergy.

To act as contact allergens, organic chemicals must form covalent bonds with skin proteins. It has been suggested that oxidation of PPD to the highly reactive chemical 1,4-benzoquinone is the probable explanation for the allergenicity.

It has also been speculated that the oxidation of PPD to its trimer (known as Bandrowski's base) may account for the allergenicity of PPD. The clinical evidence in support of this hypothesis is limited.

Alternatively, the oxidative formation of the benzoquinonediimine intermediate from PPD, which produces a very unstable and highly reactive chemical species, which would be anticipated to be highly allergenic in vivo, has been proposed.

Whatever the underlying mechanism responsible for the allergenicity of aromatic hair dye chemicals it is notable that all of the currently proposed models involve oxidation.

The present invention relies on this realization and provides methods, and compositions for reducing or eliminating the allergenicity of aromatic hair dye chemicals by inhibiting reducing or reversing their oxidation to allergenic intermediates.

Preferably, the compositions for inhibiting oxidation are applied to the skin prior to the application of the hair-dye in a pre-treatment and/or post-treatment step.

More preferably the compositions for inhibiting oxidation of aromatic hair dye chemicals are applied only to areas where the allergic reactions are most pronounced, such as the skin around the scalp margins, on the face and on the neck. By avoiding applying the pre-treatment to the scalp there is also less risk that the inhibition of oxidation of aromatic hair dye chemicals will interfere with the desired hair colouring process.

1. A method of reducing induction of contact allergy or reducing elicitation of allergic contact dermatitis of, in or on the skin in response to hair dye comprising inhibiting oxidation of the hair dye.

2. A method as claimed in claim 1 wherein oxidation is inhibited or reversed using one or more of: antioxidant, reducing agent, and a neutralising system.

3. A method as claimed in claim 2 wherein the antioxidant is selected from vitamin E and vitamin E derivatives.

4. A method as claimed in claim 2 or claim 3 wherein the antioxidant is selected from organic esters, fatty acids, vitamin E acetate and vitamin E linoleate.

5. A method as claimed in any one of claims 2 to 4 wherein the antioxidant is selected from 2-acetylhydroquinone, 4-butylresorcinol, acacia victoriae fruit extract, acer palmatum leaf extract, acetamidocaproic acid, acetyl benzoyloxy prasterone, acetyl cysteine, acetyl trihexyl citrate, adamantanyl hydroxylterephthalamide, adamantanylcarboxamido hydroxylbenzamide, adamantly methylhydroxylterephthalamide, adamantylcarboxamido methylhydroxylbenzamide, agrimonia eupatoria root extract, alchemilla vulgaris leaf extract, allantoin ascorbate, alpha-arbutin, alpinia uraiensis stalk/leaf water, aminoethanesulfinic acid, aminopropyl ascorbyl phosphate, aminopropyl tocopheryl phosphate, anserine, arabidopsis thaliana extract, arbutin, arctium lappa fruit extract, argon, asarum heterotropoides rhizome extract, ascorbic acid, ascorbic acid polypeptide, ascorbyl dipalmitate, ascorbyl glucoside, ascorbyl linoleate, ascorbyl methylsilanol pectinate, ascorbyl palmitate, ascorbyl stearate, ascorbyl tetraisopalmitate, ascorbyl tocopheryl maleate, asiaticoside, avena sativa kernel extract, bacillus/rice bran extract/soybean extract ferment filtrate, benzoguanamine, beta vulgaris root powder, bha, bht, bis-demethoxycurcumin, bis-hydroxyethyl tocopherylsuccinoylamido hydroxypropane, bromphenol blue, buddleja axillaris leaf extract, butylated xylenol, caffeic acid, calcium ascorbate, calophyllum inophyllum seed oil, camellia sinensis catechins, camellia sinensis leaf extract camellia sinensis leaf oil, capparis moonii fruit extract, capsicum annuum fruit extract, carnosic acid, carotenoids, cayaponia tayuya root extract, cercis chinensis flower/leaf/stem extract, chaenomeles sinensis fruit extract, chitosan ascorbate, chitosan glycolate, chitosan salicylate, chlorogenic acids, chrysanthemum boreale flower extract, cimicifuga dahurica root extract, cimicifuga racemosa root extract, cinnamomum zeylanicum bark extract, citrus junos seed extract, citrus medica vulgaris fruit extract, cobalt dna, copper adenosine triphosphate, coptis chinensis root extract, cordyceps sinensis extract, curcumin, cyamopsis tetragonoloba symbiosome extract, cyclopia genistoides leaf extract, cysteamine HCl, cysteine, cysteine HCl, davidsonia pruriens fruit extract, decyl mercaptomethylimidazole, demethoxycurcumin, di-t-butylhydroquinone, diacetylcurcumin, diammonium edta, diamylhydroquinone, dibenzothiophene, dibutylene tetrafurfural, dicetyl thiodipropionate, dicyclopentadiene/t-butylcresol copolymer, digalloyl trioleate, dilauryl thiodipropionate, dimethoxy di-p-cresol, dimethylmethoxy chromanol, dimyristyl thiodipropionate, dioleyl tocopheryl methylsilanol, diosmine, diospyros kaki calyx extract, diretinyl ether, disodium ascorbyl sulfate, disodium rutinyl disulfate, disodium salicylphosphate, disodium ubiquinone, distearyl thiodipropionate, ditridecyl thiodipropionate, dodecyl gallate, dunaliella bardawil powder, epigallocatechin gallate, epimedium sagittatum extract, ergothioneine, eriobotrya japonica leaf protoplast extract, eriocaulon buergarianum extract, erythorbic acid, ethyl ferulate, ethylbisiminomethylguaiacol manganese chloride, ethylhexyl ferulate, ethylhexyl gallate, eucommia ulmoides bark extract, forsythia suspensa fruit extract, fragilaria pinnata extract, furfuryl palmitate, gallic acid, ginkgo biflavones, ginkgo leaf terpenoids, glucosylrutin, glyceryl chromonyl ether, glycyrrhiza glabra root water, haematococcus pluvialis extract, haematococcus pluvialis oil, haematococcus pluvialis powder, hesperetin, hesperetin laurate, hesperidin methyl chalcone, histidine hydrochloride, honokiol, hydrolyzed aspergillus/ginseng extract ferment, hydrolyzed gardenia florida extract, hydrolyzed proanthocyanidin, hydrolyzed soy extract, hydroxy benzoxathiolone, hydroxydecyl ubiquinone, hydroxylamine HCl, hydroxylamine sulfate, inositol hexaniacinate hexaascorbate, isoquercitrin, kaempferol, kojic acid, kojyl glucoside, kojyl methylenedioxycinnamate, larrea divaricata extract, lens culinaris symbiosome extract, ligusticum striatum root extract, linseed oil ascorbate esters, lotus japonicus symbiosome extract, lycium chinense fruit extract, lycopene, lysine hydrochloride, madecassoside, magnesium ascorbate, magnesium ascorbate/pca, magnesium ascorbyl phosphate, magnolol, malachite extract, malpighia punicifolia fruit extract, manganese adenosine triphosphate, manganese dioxide, matteuccia struthiopteris extract, melaleuca alternifolia leaf oil, melatonin, melia azadirachta conditioned media/culture, methoxy PEG-7 acorbic acid, methoxytrimethylphenyl dihydroxyphenyl propanol, methyl di-t-butyl hydroxyhydrocinnamate, methylene di-t-butylcresol, methylsilanol ascorbate, murraya exotica leaf extract, niacinamide hydroxybenzoate, niacinamide thioctate, nordihydroguaiaretic acid, o-tolyl biguanide, octadecyl di-t-butyl-4-hydroxyhydrocinnamate, octanicotinoyl epigallocatechin gallate, olea europaea bud extract, olea europaea fruit unsaponifiables, oxycoccus palustris seed oil, p-hydroxyanisole, paeonol, palmitoyl grapevine shoot extract, PEG/PPG-100/70 tocopheryl ether, PEG/PPG-2/5 tocopheryl ether, PEG/PPG-30/10 tocopheryl ether, PEG/PPG-5/10 tocopheryl ether, PEG/PPG-5/20 tocopheryl ether, PEG/PPG-5/30 tocopheryl ether, PEG/PPG-50/20 tocopheryl ether, PEG/PPG-70/30 tocopheryl ether, pentaerythrityl tetra-di-t-butyl hydroxyhydrocinnamate, perilla ocymoides seed extract, perillyl alcohol, phenylthioglycolic acid, pikea robusta extract, pinus densiflora extract, pinus pinaster bark/bud extract, pinus radiata bark extract, piper nigrum seed, piper umbellatum root extract, pisum sativum symbiosome extract, platycodon grandiflorum root extract, pogostemon cablin leaf/stem extract, polygonum cuspidatum root extract, porphyridium polysaccharide, potassium ascorbyl tocopheryl phosphate, PPG-10 tocophereth-30, PPG-2 tocophereth-5, PPG-20 tocophereth-50, PPG-30 tocophereth-70, PPG-5 tocophereth-2, PPG-5 tocopheryl ether, PPG-70 tocophereth-100, propyl gallate, prunus cerasus extract, prunus cerasus fruit extract, prunus cerasus leaf extract, prunus salicina fruit extract, prunus spinosa wood extract, pyridoxine hydroxybenzoate, pyridoxine hydroxycitrate, pyridoxine salicylate, pyridoxine serinate, quercetin, quercetin caprylate, resacetophenone, resveratrol, retynil formyl aspartamate, rhodochrosite extract, rosa davurica bud extract, rosmarinic acid, rosmarinus officinalis flower extract, rubus arcticus fruit extract, rubus chamaemorus fruit extract, rutin, salnacedin, salvia officinalis extract, sambucus nigra bud extract, santalum acuminatum fruit extract, sargassum pallidum extract, sarothamnus scoparius extract, sedum rosea root extract, sideritis syriaca extract, smithsonite extract, sodium ascorbate, sodium ascorbyl phosphate, sodium ascorbyl/cholesteryl phosphate, sodium bisulfite, sodium erythorbate, sodium metabisulfite, sodium phosphono-pyridoxylidenerhodanine, sodium thioglycolate, sodium tocopheryl phosphate, sodium zinc histidine dithiooctanamide, sorbityl furfural, sponge extract, stearyl gallate, succinoyl ascorbate pentapeptide-6, superoxide dismutase,
syzygium leuhmanii fruit extract, t-butylbenzamido hydroxylbenzamide, t-butylbenzamido hydroxylphenylacetamide, t-butylbenzamido methylhydroxylbenzamide, tbhq, telmesteine, terminalia ferdinandiana fruit extract, tetrabutyl ethylidenebisphenol, tetrabutyl phenyl hydroxybenzoate, tetrahexyldecyl ascorbate, tetrahydrobisdemethoxydiferuloylmethane, tetrahydrocurcumin diacetate, tetrahydrodemethoxydiferuloylmethane, tetrahydrodiferuloylmethane, tetramethylchromanol glucosides, thioctic acid, thiodiglycol, thiodiglycolamide, thiodiglycolic acid, thioglycolic acid, thiolactic acid, thiomorpholinone, thiosalicylic acid, thiotaurine, thioxanthine, thymol trimethoxycinnamate, tococysteamide, tocophereth-10, tocophereth-12, tocophereth-18, tocophereth-5, tocophereth-50, tocopherol, tocophersolan, tocopheryl acetate, tocopheryl dimethylglycinate HCl, tocopheryl linoleate, tocopheryl linoleate/oleate, tocopheryl nicotinate, tocopheryl retinoate, tocopheryl succinate, tocoquinone, totarol, tremella fuciformis sporocarp extract, triethyl citrate, trifolium pratense leaf extract, trifolium pratense symbiosome extract, trifolium repens extract, trigonella foenum symbiosome extract, tripropylene glycol, tris(nonylphenyl)phosphite, trisodium ascorbyl isopalmitate phosphate, trisodium ascorbyl palmitate phosphate, trisodium fructose diphosphate, trisodium resveratrol triphosphate, tyrosyl histidine HCl, ubiquinol, ubiquinone, vaccinium myrtillus bud extract, vaccinium myrtillus stem extract, vaccinium vitis-idaea fruit extract, vacciniumvitis-idaea leaf protoplasts, vaccinium vitis-idaea seed oil, vicia sativa symbiosome extract, vitis vinifera fruit powder, vitis vinifera juice, vitis vinifera juice extract, vitis vinifera seed extract, vitis vinifera skin extract, vitis vinifera skin powder, xylyl dibutylbenzofuranone, zinc adenosine triphosphate, zinc ascorbate, zinc dibutyldithiocarbamate and zinc thiosalicylate.

6. A method as claimed in any one of claims 2 to 5 wherein the reducing agent is selected from aminoethanesulfinic acid, ammonium bisulfite, ammonium sulfate, ammonium sulfite, ammonium thiocyanate, ammonium thioglycolate, ammonium thiolactate, calcium thioglycolate, cysteamine HCl, cysteine cysteine HCl, dihydroxyacetone, dithiodiglycolic acid, dithiothreitol, ethanolamine dithiodiglycolate, ethanolamine thioglycolate, ethyl thioglycolate, glutathione, glyceryl thiopropionate, histidine hydrochloride, lysine hydrochloride, magnesium thioglycolate, mercaptopropionic acid, ninhydrin, p-hydroxyanisole, potassium metabisulfite, potassium sulfite, potassium, tetrathionate, sodium bisulfite, sodium hydrosulfite, sodium hydroxymethane sulfonate, sodium metabisulfite, sodium thioglycolate, sodium thiosulfate, sodium tocopheryl phosphate, stannous chloride, strontium thioglycolate, superoxide dismutase, thiodiglycolic acid, thioglycerin, thioglycolic acid, thiolactic acid, thiosalicylic acid and zinc formaldehyde sulfoxylate.

7. A method as claimed in any preceding claim wherein the skin is pre-treated to inhibit oxidation prior to the application of the hair dye

8. A method as claimed in claim 7 wherein the pre-treatment is confined to non-scalp skin.

9. A method as claimed in any preceding claim wherein the skin is post-treated to inhibit or reverse oxidation after the application of the hair dye.

10. A method as claimed in claim 9 wherein the post-treatment is confined to non-scalp skin.

11. A composition for reducing induction of contact allergy or reducing elicitation of allergic contact dermatitis of skin to hair dye comprising one or more of: antioxidants, reducing agents, and a neutralising system.

12. A composition as claimed in claim 11 wherein the antioxidant is selected from vitamin E, vitamin E derivatives, organic esters, fatty acids, vitamin E acetate, vitamin E linoleate, 2-acetylhydroquinone, 4-butylresorcinol, acacia victoriae fruit extract, acer palmatum leaf extract, acetamidocaproic acid, acetyl benzoyloxy prasterone, acetyl cysteine, acetyl trihexyl citrate, adamantanyl hydroxylterephthalamide, adamantanylcarboxamido hydroxylbenzamide, adamantly methylhydroxylterephthalamide, adamantylcarboxamido methylhydroxylbenzamide, agrimonia eupatoria root extract, alchemilla vulgaris leaf extract, allantoin ascorbate, alpha-arbutin, alpinia uraiensis stalk/leaf water, aminoethanesulfinic acid, aminopropyl ascorbyl phosphate, aminopropyl tocopheryl phosphate, anserine, arabidopsis thaliana extract, arbutin, arctium lappa fruit extract, argon, asarum heterotropoides rhizome extract, ascorbic acid, ascorbic acid polypeptide, ascorbyl dipalmitate, ascorbyl glucoside, ascorbyl linoleate, ascorbyl methylsilanol pectinate, ascorbyl palmitate, ascorbyl stearate, ascorbyl tetraisopalmitate, ascorbyl tocopheryl maleate, asiaticoside, avena sativa kernel extract, bacillus/rice bran extract/soybean extract ferment filtrate, benzoguanamine, beta vulgaris root powder, bha, bht, bis-demethoxycurcumin, bis-hydroxyethyl tocopherylsuccinoylamido hydroxypropane, bromphenol blue, buddleja axillaris leaf extract, butylated xylenol, caffeic acid, calcium ascorbate, calophyllum inophyllum seed oil, camellia sinensis catechins, camellia sinensis leaf extract camellia sinensis leaf oil, capparis moonii fruit extract, capsicum annum fruit extract, carnosic acid, carotenoids, cayaponia tayuya root extract, cercis chinensis flower/leaf/stem extract, chaenomeles sinensis fruit extract, chitosan ascorbate, chitosan glycolate, chitosan salicylate, chlorogenic acids, chrysanthemum boreale flower extract, cimicifuga dahurica root extract, cimicifuga racemosa root extract, cinnamomum zeylanicum bark extract, citrus junos seed extract, citrus medica vulgaris fruit extract, cobalt dna, copper adenosine triphosphate, coptis chinensis root extract, cordyceps sinensis extract, curcumin, cyamopsis tetragonoloba symbiosome extract, cyclopia genistoides leaf extract, cysteamine HCl, cysteine, cysteine HCl, davidsonia pruriens fruit extract, decyl mercaptomethylimidazole, demethoxycurcumin, di-t-butylhydroquinone, diacetylcurcumin, diammonium edta, diamylhydroquinone, dibenzothiophene, dibutylene tetrafurfural, dicetyl thiodipropionate, dicyclopentadiene/t-butylcresol copolymer, digalloyl trioleate, dilauryl thiodipropionate, dimethoxy di-p-cresol, dimethylmethoxy chromanol, dimyristyl thiodipropionate, dioleyl tocopheryl methylsilanol, diosmine, diospyros kaki calyx extract, diretinyl ether, disodium ascorbyl sulfate, disodium rutinyl disulfate, disodium salicylphosphate, disodium ubiquinone, distearyl thiodipropionate, ditridecyl thiodipropionate, dodecyl gallate, dunaliella bardawil powder, epigallocatechin gallate, epimedium sagittatum extract, ergothioneine, eriobotrya japonica leaf protoplast extract, eriocaulon buergarianum extract, erythorbic acid, ethyl ferulate, ethylbisiminomethylguaiacol manganese chloride, ethylhexyl ferulate, ethylhexyl gallate, eucommia ulmoides bark extract, forsythia suspensa fruit extract, fragilaria pinnata extract, furfuryl palmitate, gallic acid, ginkgo biflavones, ginkgo leaf terpenoids, glucosylrutin, glyceryl chromonyl ether, glycyrrhiza glabra root water, haematococcus pluvialis extract, haematococcus pluvialis oil, haematococcus pluvialis powder, hesperetin, hesperetin laurate, hesperidin methyl chalcone, histidine hydrochloride, honokiol, hydrolyzed aspergillus/ginseng extract ferment, hydrolyzed gardenia florida extract, hydrolyzed proanthocyanidin, hydrolyzed soy extract, hydroxy benzoxathiolone, hydroxydecyl ubiquinone, hydroxylamine HCl, hydroxylamine sulfate, inositol hexaniacinate hexaascorbate, isoquercitrin, kaempferol, kojic acid, kojyl glucoside, kojyl methylenedioxycinnamate, larrea divaricata extract, lens culinaris symbiosome extract, ligusticum striatum root extract, linseed oil ascorbate esters, lotus japonicus symbiosome extract, lycium chinense fruit extract, lycopene, lysine hydrochloride, madecassoside, magnesium ascorbate, magnesium ascorbate/pca, magnesium ascorbyl phosphate, magnolol, malachite extract, malpighia punicifolia fruit extract, manganese adenosine triphosphate, manganese dioxide, matteuccia struthiopteris extract, melaleuca alternifolia leaf oil, melatonin, melia azadirachta conditioned media/culture, methoxy PEG-7 acorbic acid, methoxytrimethylphenyl dihydroxyphenyl propanol, methyl di-t-butyl hydroxyhydrocinnamate, methylene di-t-butylcresol, methylsilanol ascorbate, murraya exotica leaf extract, niacinamide hydroxybenzoate, niacinamide thioctate, nordihydroguaiaretic acid, o-tolyl biguanide, octadecyl di-t-butyl-4-hydroxyhydrocinnamate, octanicotinoyl epigallocatechin gallate, olea europaea bud extract, olea europaea fruit unsaponifiables, oxycoccus palustris seed oil, p-hydroxyanisole, paeonol, palmitoyl grapevine shoot extract, PEG/PPG-100/70 tocopheryl ether, PEG/PPG-2/5 tocopheryl ether, PEG/PPG-30/10 tocopheryl ether, PEG/PPG-5/10 tocopheryl ether, PEG/PPG-5/20 tocopheryl ether, PEG/PPG-5/30 tocopheryl ether, PEG/PPG-50/20 tocopheryl ether, PEG/PPG-70/30 tocopheryl ether, pentaerythrityl tetra-di-t-butyl hydroxyhydrocinnamate, perilla ocymoides seed extract, perillyl alcohol, phenylthioglycolic acid, pikea robusta extract, pinus densiflora extract, pinus pinaster bark/bud extract, pinus radiata bark extract, piper nigrum seed, piper umbellatum root extract, pisum sativum symbiosome extract, platycodon grandiflorum root extract, pogostemon cablin leaf/stem extract, polygonum cuspidatum root extract, porphyridium polysaccharide, potassium ascorbyl tocopheryl phosphate, PPG-10 tocophereth-30, PPG-2 tocophereth-5, PPG-20 tocophereth-50, PPG-30 tocophereth-70, PPG-5 tocophereth-2,
PPG-5 tocopheryl ether, PPG-70 tocophereth-100, propyl gallate, prunus cerasus extract, prunus cerasus fruit extract, prunus cerasus leaf extract, prunus salicina fruit extract, prunus spinosa wood extract, pyridoxine hydroxybenzoate, pyridoxine hydroxycitrate, pyridoxine salicylate, pyridoxine serinate, quercetin, quercetin caprylate, resacetophenone, resveratrol, retynil formyl aspartamate, rhodochrosite extract, rosa davurica bud extract, rosmarinic acid, rosmarinus officinalis flower extract, rubus arcticus fruit extract, rubus chamaemorus fruit extract, rutin, salnacedin, salvia officinalis extract, sambucus nigra bud extract, santalum acuminatum fruit extract, sargassum pallidum extract, sarothamnus scoparius extract, sedum rosea root extract, sideritis syriaca extract, smithsonite extract, sodium ascorbate, sodium ascorbyl phosphate, sodium ascorbyl/cholesteryl phosphate, sodium bisulfite, sodium erythorbate, sodium metabisulfite, sodium phosphono-pyridoxylidenerhodanine, sodium thioglycolate, sodium tocopheryl phosphate, sodium zinc histidine dithiooctanamide, sorbityl furfural, sponge extract, stearyl gallate, succinoyl ascorbate pentapeptide-6, superoxide dismutase, syzygium leuhmanii fruit extract, t-butylbenzamido hydroxylbenzamide, t-butylbenzamido hydroxylphenylacetamide, t-butylbenzamido methylhydroxylbenzamide, tbhq, telmesteine, terminalia ferdinandiana fruit extract, tetrabutyl ethylidenebisphenol, tetrabutyl phenyl hydroxybenzoate, tetrahexyldecyl ascorbate, tetrahydrobisdemethoxydiferuloylmethane, tetrahydrocurcumin diacetate, tetrahydrodemethoxydiferuloylmethane, tetrahydrodiferuloylmethane, tetramethylchromanol glucosides, thioctic acid, thiodiglycol, thiodiglycolamide, thiodiglycolic acid, thioglycolic acid, thiolactic acid, thiomorpholinone, thiosalicylic acid, thiotaurine, thioxanthine, thymol trimethoxycinnamate, tococysteamide, tocophereth-10, tocophereth-12, tocophereth-18, tocophereth-5, tocophereth-50, tocopherol, tocophersolan, tocopheryl acetate, tocopheryl dimethylglycinate HCl, tocopheryl linoleate, tocopheryl linoleate/oleate, tocopheryl nicotinate, tocopheryl retinoate, tocopheryl succinate, tocoquinone, totarol, tremella fuciformis sporocarp extract, triethyl citrate, trifolium pratense leaf extract, trifolium pratense symbiosome extract, trifolium repens extract, trigonella foenum symbiosome extract, tripropylene glycol, tris(nonylphenyl)phosphite, trisodium ascorbyl isopalmitate phosphate, trisodium ascorbyl palmitate phosphate, trisodium fructose diphosphate, trisodium resveratrol triphosphate, tyrosyl histidine HCl, ubiquinol, ubiquinone, vaccinium myrtillus bud extract, vaccinium myrtillus stem extract, vaccinium vitis-idaea fruit extract, vacciniumvitis-idaea leaf protoplasts, vaccinium vitis-idaea seed oil, vicia sativa symbiosome extract, vitis vinifera fruit powder, vitis vinifera juice, vitis vinifera juice extract, vitis vinifera seed extract, vitis vinifera skin extract, vitis vinifera skin powder, xylyl dibutylbenzofuranone, zinc adenosine triphosphate, zinc ascorbate, zinc dibutyldithiocarbamate and zinc thiosalicylate.

13. A composition as claimed in any one of claims 11 to 12 wherein the reducing agent is selected from aminoethanesulfinic acid, ammonium bisulfite, ammonium sulfate, ammonium sulfite, ammonium thiocyanate, ammonium thioglycolate, ammonium thiolactate, calcium thioglycolate, cysteamine HCl, cysteine cysteine HCl, dihydroxyacetone, dithiodiglycolic acid, dithiothreitol, ethanolamine dithiodiglycolate, ethanolamine thioglycolate, ethyl thioglycolate, glutathione, glyceryl thiopropionate, histidine hydrochloride, lysine hydrochloride, magnesium thioglycolate, mercaptopropionic acid, ninhydrin, p-hydroxyanisole, potassium metabisulfite, potassium sulfite, potassium, tetrathionate, sodium bisulfite, sodium hydrosulfite, sodium hydroxymethane sulfonate, sodium metabisulfite, sodium thioglycolate, sodium thiosulfate, sodium tocopheryl phosphate, stannous chloride, strontium thioglycolate, superoxide dismutase, thiodiglycolic acid, thioglycerin, thioglycolic acid, thiolactic acid, thiosalicylic acid and zinc formaldehyde sulfoxylate.
